(19) United States
(12) Patent Application Publication
(10) Pub. No.: US 2024/0335322 A1
(43) Pub. Date: Oct. 10, 2024

(54) METHOD FOR PROVIDING CONTROL DATA FOR AN OPHTHALMOLOGICAL LASER OF A TREATMENT APPARATUS FOR CORRECTING A CORNEA

(71) Applicant: SCHWIND EYE-TECH-SOLUTIONS GMBH, Kleinostheim (DE)

(72) Inventor: Samuel ARBA MOSQUERA, Aschaffenburg (DE)

(21) Appl. No.: 18/626,497

(22) Filed: Apr. 4, 2024

(30) Foreign Application Priority Data
Apr. 4, 2023 (DE) 102023108625.1

(51) Int. Cl. A61F 9/008; G16H 20/40; G16H 40/63

(52) U.S. Cl. CPC A61F 9/00804; A61F 9/00827; G16H 20/40; G16H 40/63; A61F 2009/00872; A61F 2009/0088; A61F 2009/00882

(57) ABSTRACT

The invention relates to a system and method for providing control data for an ophthalmological laser of a treatment apparatus for correcting a cornea. The method includes ascertaining topographic data of the preoperative cornea from predetermined examination data; calculating wavefront aberration data of the preoperative cornea by the topographic data, wherein a passage of light beams through the cornea, which has the topographic data, is determined by a beam passage model for calculating the wavefront aberration data; ascertaining an aberration-neutral correction profile, by which higher order aberrations of the preoperative cornea are preserved for a postoperative cornea, wherein a predetermined refraction correction is adapted depending on the ascertained wavefront aberration data for ascertaining the aberration-neutral correction profile; and providing the control data for correcting the cornea for the ophthalmological laser, which includes the aberration-neutral correction profile.

### FIELD

The invention relates to a method for providing control data for an ophthalmological laser of a treatment apparatus for correcting a cornea. In addition, the invention relates to a treatment apparatus with at least one eye surgical laser and at least one control device for performing the method, to a computer program and to a computer-readable medium.

### BACKGROUND

Treatment apparatuses and methods for controlling ophthalmological lasers for correcting an optical visual disorder and/or pathologically or unnaturally altered areas of the cornea are known in the prior art. Therein, pulsed lasers and a beam focusing device can for example be formed such that laser pulses effect a photodisruption and/or ablation in a focus situated within the organic tissue to remove a tissue, in particular a tissue lenticule, from the cornea.

Upon corrections on the cornea, in particular upon aspherical corrections, however, it can occur that undesired higher order aberrations are induced by the treatment. In particular, spherical aberrations can arise upon a correction.

Therefore, it is the object of the invention to provide control data for correcting a cornea, in which the development of higher order aberrations is reduced or avoided.

### SUMMARY

This object is solved by the independent claims. Advantageous embodiments are disclosed in the dependent claims, the following description as well as the figures.

The techniques described herein are based on the idea that higher order aberrations, which a cornea has, are ascertained before the treatment and the postoperative cornea is planned such that these aberrations are preserved and new aberrations are not generated. Hereto, the aberrations of the cornea are modeled by a beam passage model and the correction profile for a preset refraction correction is adapted such that these aberrations remain constant for the postoperative cornea.

According to an aspect, a method for providing control data for an ophthalmological laser of a treatment apparatus for correcting a cornea is described. The method can be performed by a control device or the control data can be planned by the control device, wherein an appliance or an appliance component, in particular a processor or microprocessor, is meant by a control device, by which the following method steps can be performed.

Ascertaining topographic data of the preoperative cornea from predetermined examination data, and calculating wavefront aberration data of the preoperative cornea by the topographic data are effected, wherein a passage of light beams through the cornea, which has the topographic data, is determined by a beam passage model for calculating the wavefront aberration data. Furthermore, ascertaining an aberration-neutral correction profile is effected, by which higher order aberrations of the preoperative cornea are preserved for a postoperative cornea, wherein a predetermined refraction correction is adapted depending on the ascertained wavefront aberration data for ascertaining the aberration-neutral correction profile. Finally, providing the control data for correcting the cornea for the ophthalmological laser, which includes the aberration-neutral correction profile, is effected.

In other words, topographic data, such as for example a corneal curvature or a topography, is determined in particular for an aspherical correction. Hereto, predetermined examination data can be present, such as for example from a videokeratoscope, to obtain the topographic data. Then, aberrations of a wavefront can be calculated from the topographic data, which arise upon passage through the cornea, wherein the aberrations can be provided as wavefront aberration data. For example, the wavefront aberration data can be described by Zernike polynomials, by which lower and higher order aberrations can be formulated.

Therein, the calculation of the wavefront aberration data can be performed by a beam passage model, in which a deflection of light beams by the cornea, which has the ascertained topography, is modeled. For example, the beam passage model can be based on a ray tracing method, on a Fermat method and/or on a method of surface aberrations.

A predetermined refraction correction for refractive power change of the cornea can then be adapted such that the postoperative cornea does not have new or changed higher order aberrations compared to the preoperative cornea. This means that a correction profile, in particular an ablation profile and/or a lenticule geometry, is changed, which can be ascertained from the predetermined refraction correction, and not initially determined diopter values, which are used for this correction. Thus, the cornea is kept aberration-neutral for higher order aberrations before and after the treatment within an optical zone of the treatment. The refraction correction used thereto can for example be predetermined from a subjective correction or glasses correction by a phoropter.

For example, in order to ascertain how the refraction correction is to be adapted to obtain the aberration-neutral correction profile, virtual topographic data of a virtual postoperative cornea can be modeled, from which virtual wavefront aberration data may also be calculated by the beam passage model. This means, it can be modeled how the cornea looks like after performing the refraction correction by the correction profile, wherein aberrations can be subsequently ascertained for this postoperative cornea. Subsequently, a difference can be ascertained between the aberrations, which are present in the preoperative cornea, in particular the higher order aberrations, and the aberrations, which are present in the postoperative cornea, wherein the correction profile is adapted by this difference to obtain the aberration-neutral correction profile.

For example, both the curvature of the cornea before the treatment, in particular the “keratometry readings”, and a refraction, which is to be performed, can be known. Thereby, the postoperative keratometry readings to be expected can be ascertained, wherein the ascertained higher order aberrations, which are to be kept constant by the treatment, can be imputed to the postoperative cornea to determine the aberration-neutral correction profile from it. In a further example, the higher order aberrations can be ascertained by the beam passage model from the geometry/topography of the cornea. From the predetermined refraction, which results in the correction profile for removing the tissue, the postoperative geometry/topography of the cornea to be expected can then be ascertained, wherein the expectable postoperative higher aberrations of the postoperative geometry/topography can also be determined by the beam passage model. From the difference of the preoperative and postoperative higher order aberrations, the higher order aberrations can then be determined, which have to be added to the correction profile to obtain them upon the treatment.

In particular, it can also be provided that upon removal of unnaturally changed tissue or scars in the cornea, lower order aberrations are also kept neutral besides the higher order aberrations. Thus, the tissue can be removed without a refractive power change occurring and/or higher order aberrations being generated.

Finally, after determining the aberration-neutral correction profile, control data for correcting the cornea can be provided for the ophthalmological laser and/or the treatment apparatus, which includes the aberration-neutral correction profile. By the control data, the laser and/or the treatment apparatus can then be controlled without inducing additional aberrations. The control data can include a respective dataset for positioning and/or for focusing individual laser pulses in the cornea. Additionally or alternatively, a respective dataset for adjusting at least one beam device for beam guidance and/or beam shaping and/or beam deflection and/or beam focusing of a laser beam of the respective laser can be included in the control data.

According to the techniques described herein, the advantage arises that improved control data for controlling the ophthalmological laser can be provided since the formation of new aberrations, in particular higher order aberrations, can be avoided.

This description includes exemplary embodiments, by which additional advantages arise.

In an embodiment, the beam passage model is based on the ray tracing method, in which the refraction of light beams by the cornea is modeled with the ascertained topographic data according to the Snell's law. This means that the calculation of wavefront aberration data can be ascertained in discrete points on an optical surface, which can be provided by the topographic data, by ray tracing. In the ray tracing method, a corneal surface and a pupil can be considered isolated from the remainder of the eye. With the aid of the ray tracing method, the optical wavefront, which propagates through the pupil after refraction on the cornea, can be mathematically constructed based on the Snell's law. In case of a theoretically aberration-free cornea, the wavefront is spherical, wherein the radius is centered in the focal point. This “pupil sphere” is the reference surface, from which the wavefront aberrations can be determined. If aberrations are present due to the shape of the corneal surface, the actual wavefront deviates from the pupil sphere and the distance of the optical path between these surfaces is the wavefront aberration, which can be provided in the form of the wavefront aberration data. Therein, the distance can be measured along the refracted beam. By multiplication by a refractive index, this physical distance is converted into an optical path distance, which can be expressed in micrometers or wavelengths. In the ray tracing method, the propagation of the light beams is calculated according to the Snell's law, which describes the direction change of a propagation direction of light beams or plane waves upon transition into a different medium due to a changed, material-dependent phase velocity, which is included in the law of refraction as the refractive index. By this embodiment, an advantageous configuration of the beam passage model can be provided.

In a further embodiment, the beam passage model is based on the Fermat's principle, in which a path of the light beams through the cornea with the ascertained topographic data is modeled based on the shortest time, which a respective light beam takes through the cornea. This means that the deflection of the light beams can be modeled by the beam passage model based on the shortest time, which they take through the cornea, which includes the topographic data. In particular if light enters a medium with a different refractive index, the shortest time, which light takes for a distance between two points, cannot necessarily occur on a straight line between the points. The shortest time is rather present on the shortest optical path, which is defined as the physical distance multiplied by the refractive index. In an aberration-free optical system, two beams, which emanate from a point source and pass through the optical system, would be incident on an image point at the same time and in phase. That is, the optical path length for a peripheral and an axial beam has to be identical and the difference between their optical paths is zero. If the two beam paths are not equally long, the two light beams do not arrive in phase at the image point, which indicates that an aberration or phase shift has occurred anywhere on the path. In case of a single refracting surface like the corneal surface, thus, the phase shift can be ascribed to the shape of the corneal surface. Thus, the wavefront aberrations can for example be described as a difference of the path lengths between a reference beam, in particular an axial beam, and the further beams, which traverse the surface. Thus, an optical path between a front and a rear corneal surface can in particular also be calculated to estimate wavefront aberrations for the entire cornea. By this embodiment, a further advantageous configuration of the beam passage model can be provided.

In yet a further embodiment, the beam passage model is based on a surface aberration method, in which a wavefront aberration is modeled to a Cartesian oval by a difference of a corneal curvature, which is provided from the topographic data. Herein, it is assumed that optical path differences, which cause aberrations, arise by the shape of the corneal surface. Thus, wavefront aberrations of the cornea can be directly calculated from the topographic data. In particular in case of a rotationally symmetric cornea, a surface shape, which is free of aberrations, is an ellipsoid with a preset eccentricity, which is equal to a reciprocal value of the refractive index. This is also referred to as Cartesian oval. Each deviation from this surface results in aberrations. The relative elevation of the cornea compared to the Cartesian oval is thus a “surface aberration”. If it is known, the wavefront aberration of the cornea can be derived from this geometry. With an aberration-free cornea, a beam from a distant axial point can in particular be refracted in a point and propagate towards the focal point. With a real cornea with a surface shape other than the Cartesian oval, an optical distance traveled between the object and the image point is deviating compared to the distance traveled in the aberration-free case. The difference between the aberration-free path through the Cartesian oval and the path traveled through the real cornea is the wavefront aberration. Thus, the wavefront aberration can be calculated by the surface aberration multiplied by n−1. By this embodiment, a further advantageous configuration of the beam passage model can be provided.

A further embodiment provides that for determining the aberration-neutral correction profile, virtual topographic data of a virtual postoperative cornea, which has been treated by the predetermined refraction correction, is modeled, wherein virtual wavefront aberration data for the virtual postoperative cornea is calculated by the virtual topographic data and the beam passage model, wherein the predetermined refraction correction is adapted by a difference between the virtual wavefront aberration data of the virtual postoperative cornea and the wavefront aberration data of the preoperative cornea for providing the aberration-neutral correction profile. In other words, it can be provided that a virtual postoperative cornea is modeled in that a treatment of the preoperative cornea by the predetermined refraction correction is simulated. For the virtual postoperative cornea, it can then be checked in similar manner as for the preoperative cornea, which wavefront aberrations, in particular higher order aberrations, are present. They can then be compared to the wavefront aberrations, which have been determined in the preoperative cornea, wherein a difference therefrom can be added to a correction profile of the refraction correction to keep the higher order aberrations constant before and after the treatment. Hereby, an advantageous variant for ascertaining the aberration-neutral correction profile can be provided.

In a further embodiment, a reference center is set for the topographic data, which is defined by a point of intersection of an axis of vision with a corneal surface, wherein the axis of vision extends from a central point of a pupil up to a fixation point external to eye. This means that a reference center can be set, based on which corneal elevations and thus the wavefront aberrations can be determined, wherein a point of intersection of an axis of vision with the cornea is advantageously defined as the reference center. Therein, the axis of vision can be defined by a line, which extends from a pupil center on a line of sight to a fixation point external to eye. In order to correctly calculate wavefront aberrations of the cornea from topographic data, such as for example videokeratoscopic data, it can in particular be determined, around which corneal reference point and which reference axis the corneal elevations are measured. Herein, different points or reference axes of the eye can be defined as the reference center, such as for example the axis of vision, a visual axis (foveal achromatic axis), a pupil axis or an optical axis of the cornea. In particular, the axis of vision can be provided as the reference center, which is also referred to as fixation axis, and represents a line, which connects the central point of the entrance pupil to a fixation point. The axis of vision is the principal beam of the light bundle, which emanates from the fixation point, is delimited by the pupil and ends in the fovea after passage through the optics of the eye. In determining the topographic data by a videokeratoscope, a patient fixes light, which is in the center of the Placido's disk pattern, wherein an optical axis of the instrument is perpendicular to the cornea. Therefore, the optical axis of the instrument is aligned with the center of the corneal curvature, but which does not necessarily pass through the center of the entrance pupil. In most cases, the central point of the entrance pupil is slightly shifted from the axis of the videokeratoscope, and the line of sight is shifted from the axis of the videokeratoscope. The point, in which the line of sight intersects the corneal surface, is referred to as corneal sighting center. Now, if the Fermat method is for example used for calculating the wavefront aberration, the topographic data of the cornea can be mathematically centered on the line of sight or axis of vision in that a reference beam is defined as that beam, which passes from a distant object point on the line of sight through the corneal sighting center and finally ends in the image point. Further beams can be drawn through other corneal points scanned by the videokeratoscope between the object and the image point. Optical path differences between the reference beam and all of the other beams, which intersect the cornea, thus define the wavefront aberrations, which are connected to these points of intersection. Accordingly, a determination of wavefront aberration data by the beam passage model can be simplified by setting a suitable reference center.

In a further embodiment, predetermined corneal tomography data is used for calculating the wavefront aberration data. Thus, anterior and posterior corneal surface data can be ascertained, which describes a geometry of the cornea, in particular also on a posterior surface of the cornea. Thus, a determination of the wavefront aberration data can be improved by the beam passage model.

In a further embodiment, predetermined ocular aberration data is additionally used for the aberration-neutral correction profile. In other words, not only wavefront aberration data of the cornea, but of the entire eye can be taken into account. This ocular aberration data can for example be determined by an aberrometer or a wavefront measurement of the eye. This ocular aberration data can then be included in the calculation of the aberration-neutral correction profile to overall obtain an aberration-free correction profile, which does not have higher order aberrations anymore. Hereby, the advantage arises that the higher order aberrations are not only kept constant before and after the treatment, but aberrations can be removed.

In another aspect, a method for controlling a treatment apparatus is described. The method includes the method steps of at least one embodiment of a method as it was previously described. Furthermore, the method for controlling the treatment apparatus also includes the step of transferring the provided control data to at least one ophthalmological laser of the treatment apparatus and controlling the treatment apparatus and/or the laser with the control data.

In some instances, the respective method can include at least one additional step, which is executed if and only if an application case or an application situation occurs, which has not been explicitly described here. For example, the step can include the output of an error message and/or the output of a request for inputting a user feedback. Additionally or alternatively, it can be provided that a default setting and/or a predetermined initial state are adjusted.

A further aspect of the invention relates to a control device, which is formed to perform the steps of at least one embodiment of one or both of the previously described methods. Thereto, the control device can comprise a computing unit for electronic data processing such as for example a processor. The computing unit can include at least one microcontroller and/or at least one microprocessor. The computing unit can be configured as an integrated circuit and/or microchip. Furthermore, the control device can include an (electronic) data memory or a storage unit. A program code can be stored on the data memory, by which the steps of the respective embodiment of the respective method are encoded. The program code can include the control data for the respective laser. The program code can be executed by the computing unit, whereby the control device is caused to execute the respective embodiment. The control device can be formed as a control chip or control unit. The control device can for example be encompassed by a computer or computer cluster.

A further aspect of the invention relates to a treatment apparatus with at least one eye surgical or ophthalmological laser and a control device, which is formed to perform the steps of at least one embodiment of one or both of the previously described methods. The respective laser can be formed to at least partially separate a predefined corneal volume with predefined interfaces of a human or animal eye by optical breakthrough, in particular at least partially separate it by photodisruption and/or to ablate corneal layers by (photo) ablation and/or to effect a laser-induced refractive index change in the cornea and/or the eye lens and/or to increase a crosslinking of the cornea.

In a further configuration of the treatment apparatus according to the invention, the laser can be suitable to emit laser pulses in a wavelength range between 300 nm and 1400 nm, for example between 900 nm and 1200 nm, at a respective pulse duration between 1 fs and 1 ns, for example between 10 fs and 10 ps, and a repetition frequency of greater than 10 kilohertz (kHz), for example between 100 kHz and 100 megahertz (MHz). The use of such lasers in the method according to the invention additionally has the advantage that the irradiation of the cornea does not have to be effected in a wavelength range below 300 nm. This range is subsumed by the term “deep ultraviolet” in the laser technology. Thereby, it is advantageously avoided that an unintended damage to the cornea is effected by these very short-wavelength and high-energy beams. Photodisruptive and/or ablative lasers of the type used here usually input pulsed laser radiation with a pulse duration between 1 fs and 1 ns into the corneal tissue. Thereby, the power density of the respective laser pulse required for the optical breakthrough can be spatially narrowly limited such that a high incision accuracy is allowed in the generation of the interfaces. In particular, the range between 700 nm and 780 nm can also be selected as the wavelength range.

In a further embodiment of the treatment apparatus according to the invention, the control device can comprise at least one storage device for at least temporary storage of at least one control dataset, wherein the control dataset or datasets include(s) control data for positioning and/or for focusing individual laser pulses in the cornea; and can comprise at least one beam device for beam guidance and/or beam shaping and/or beam deflection and/or beam focusing of a laser beam of the laser.

A further aspect of the invention relates to a computer program. The computer program includes commands, which for example form a program code. The program code can include at least one control dataset with the respective control data for the respective laser. Upon execution of the program code by a computer or a computer cluster, it is caused to execute the previously described method or at least one embodiment thereof.

A further aspect of the invention relates to a computer-readable medium (storage medium), on which the above mentioned computer program and the commands thereof, respectively, are stored. For executing the computer program, a computer or a computer cluster can access the computer-readable medium and read out the content thereof. The storage medium is for example formed as a data memory, in particular at least partially as a volatile or a non-volatile data memory. A non-volatile data memory can be a flash memory and/or an SSD (solid state drive) and/or a hard disk. A volatile data memory can be a RAM (random access memory). For example, the commands can be present as a source code of a programming language and/or as assembler and/or as a binary code.

Further features and advantages of one of the described aspects of the invention can result from the embodiments of another one of the aspects of the invention. Thus, the features of the embodiments of the invention can be present in any combination with each other if they have not been explicitly described as mutually exclusive.

### BRIEF DESCRIPTION OF THE DRAWINGS

In the following, additional features and advantages of the invention are described in the form of advantageous execution examples based on the figure(s). The features or feature combinations of the execution examples described in the following can be present in any combination with each other and/or the features of the embodiments. This means, the features of the execution examples can supplement and/or replace the features of the embodiments and vice versa. Thus, configurations are also to be regarded as encompassed and disclosed by the invention, which are not explicitly shown or explained in the figures, but arise from and can be generated by separated feature combinations from the execution examples and/or embodiments. Thus, configurations are also to be regarded as disclosed, which do not comprise all of the features of an originally formulated claim or extend beyond or deviate from the feature combinations set forth in the relations of the claims. To the execution examples, there shows:

FIG. 1 depicts a schematic representation of a treatment apparatus according to an exemplary embodiment;

FIG. 2 depicts a flowchart for a method diagram for providing control data according to an exemplary embodiment.

In the figures, identical or functionally identical elements are provided with the same reference characters.

### DETAILED DESCRIPTION

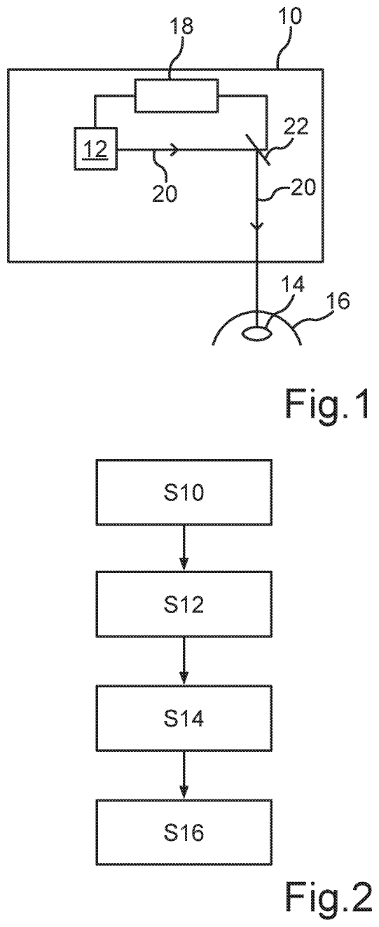

FIG. 1 shows a schematic representation of a treatment apparatus 10 with an ophthalmological laser 12 for removing a tissue 14 from a human or animal cornea 16 by photodisruption and/or ablation. For example, the tissue 14 can represent a lenticule or also volume body, which can be separated from the cornea 16 by the eye surgical laser 12 for correcting a visual disorder. A correction profile or a geometry of the tissue 14 to be removed can be provided by a control device 18, in particular in the form of control data, such that the laser 12 emits pulsed laser pulses in a pattern predefined by the control data into the cornea 16 of the eye to remove the tissue 14. Alternatively, the control device 18 can be a control device 18 external with respect to the treatment apparatus 10.

Furthermore, FIG. 1 shows that the laser beam 20 generated by the laser 12 can be deflected towards the cornea 16 by a beam deflection device 22, such as for example a rotation scanner, to remove the tissue 14. The beam deflection device 22 can also be controlled by the control device 18 to remove the tissue 14.

In particular, the illustrated laser 12 can be a photodisruptive and/or photoablative laser, which is formed to emit laser pulses in a wavelength range between 300 nanometers and 1400 nanometers, for example between 700 nanometers and 1200 nanometers, at a respective pulse duration between 1 femtosecond and 1 nanosecond, for example between 10 femtoseconds and 10 picoseconds, and a repetition frequency of greater than 10 kilohertz, for example between 100 kilohertz and 100 megahertz. In addition, the control device 18 optionally comprises a storage device (not illustrated) for at least temporary storage of at least one control dataset, wherein the control dataset or datasets include(s) control data for positioning and/or for focusing individual laser pulses in the cornea.

In removing the tissue 14 from the cornea 16, it can occur that higher order aberrations additionally arise by a changed corneal geometry, which are undesired. In order to avoid these higher order aberrations, the method shown in FIG. 2 can be performed by the control device 18 and/or by a control device external to the treatment apparatus 10, which for example belongs to a planning device.

In FIG. 2, a schematic method diagram for providing control data for the ophthalmological laser 12 of the treatment apparatus 10 is illustrated. The control data can be provided for a correction, in particular an aspherical correction of the cornea 16.

In a step S10, topographic data of the preoperative cornea 16 can be determined from predetermined examination data. The topographic data can in particular include a corneal curvature and/or a topography, which can for example be ascertained from a videokeratoscope measurement. In particular, tomography data, which is for example determined by an optical coherence tomography measurement, can also be used to obtain the topographic data of the preoperative cornea 16, wherein it can then for example include a geometry of an anterior and posterior corneal surface.

In a step S12, wavefront aberration data can be calculated from the topographic data of the preoperative cornea 16, wherein the wavefront aberration data includes higher order aberrations or wavefront aberrations, which the cornea 16 has before the treatment. For calculating the wavefront aberration data, it can be simulated by a beam passage model, how light beams change after passage through the cornea 16, which has the topographic data.

Hereto, a ray tracing method can for example be used as the beam passage model, in which light beams experience a deflection of direction upon the transition into another medium, here for example from air to corneal tissue. Thus, the respective change can be ascertained for multiple light beams, which pass the cornea 16, whereby the wavefront aberration data results. Alternatively, the beam passage model can be based on the Fermat's principle, by which the light beams are modeled based on the shortest time, which they take through the cornea 16, wherefrom the wavefront aberration data can be determined. A further alternative of the beam passage model is a surface aberration method, wherein hereto a corneal curvature, which can be ascertained from the topographic data of the cornea 16, can be compared to a curvature of a Cartesian oval, wherein the Cartesian oval represents an aberration-free deflection. By the difference to this Cartesian oval, the formation of aberrations can then be estimated.

Furthermore, a suitable reference center may be defined, based on which the topographic data and thus the wavefront aberration data is oriented. As a particularly suitable reference center, therein, the point of intersection of an axis of vision with the corneal surface has turned out, wherein the axis of vision extends from a central point of a pupil up to a fixation point external to eye. Therein, the axis of vision and thus the point of intersection of the axis of vision with the cornea can be determined during a videokeratoscope measurement.

In a step S14, an aberration-neutral correction profile can subsequently be determined, wherein by a treatment by the aberration-neutral correction profile, new higher order aberrations are not generated. In other words, by the aberration-neutral correction profile, higher order aberrations, which have been present in the preoperative cornea 16, also remain the same in the postoperative cornea, which has been treated by the aberration-neutral correction profile. Thus, only a predetermined refraction correction can in particular be planned and performed and further aberration characteristics of the cornea 16 are preserved, which results in a minimum readjustment of a patient after the eye treatment.

In order to obtain the aberration-neutral correction profile, a predetermined or planned refraction correction can be adapted such that the ascertained wavefront aberration data remain the same for the preoperative cornea 16 and the postoperative cornea. Hereto, a virtual postoperative cornea, which is expected by the predetermined refraction correction, may be modeled. From the virtual postoperative cornea, virtual topographic data can then be determined, which for example indicates, which geometry the virtual postoperative cornea has. By the beam passage model, virtual wavefront aberration data can then be calculated for the virtual topographic data. If the virtual wavefront aberration data deviates from the wavefront aberration data of the preoperative cornea 16, it is provided that the planned refraction correction is adapted by the difference, such that the wavefront aberration data is the same before and after the treatment. Therein, the adapted refraction correction represents the aberration-neutral correction profile.

Finally, control data for correcting the cornea 16, which includes the aberration-neutral correction profile, can be provided for the treatment apparatus 10 and/or the ophthalmological laser 12 in a step S16. Then, the treatment apparatus 10 can be controlled by the provided control data without inducing additional higher order aberrations in the cornea 16.

Overall, the examples show, how aberration-neutral correction profiles can be provided.

1. A method for providing control data for an ophthalmological laser of a treatment apparatus for correcting a cornea, wherein the method comprises the following steps performed by a control device:
ascertaining topographic data of the preoperative cornea from predetermined examination data;
calculating wavefront aberration data of the preoperative cornea by the topographic data, wherein a passage of light beams through the cornea, which has the topographic data, is determined by a beam passage model for calculating the wavefront aberration data;
ascertaining an aberration-neutral correction profile, by which higher order aberrations of the preoperative cornea are preserved for a postoperative cornea, wherein a predetermined refraction correction is adapted depending on the ascertained wavefront aberration data for ascertaining the aberration-neutral correction profile; and
providing the control data for correcting the cornea for the ophthalmological laser, which includes the aberration-neutral correction profile.

2. The method according to claim 1, wherein the beam passage model is based on the ray tracing method, in which the refraction of light beams by the cornea with the ascertained topographic data is modeled according to the Snell's law.

3. The method according to claim 1, wherein the beam passage model is based on the Fermat's principle, in which a path of the light beams through the cornea with the ascertained topographic data is modeled based on the shortest time, which a respective light beam takes through the cornea.

4. The method according to claim 1, wherein the beam passage model is based on a surface aberration method, in which a wavefront aberration is modeled by a difference of a corneal curvature, which is provided from the topographic data, to a Cartesian oval.

5. The method according to claim 1, wherein virtual topographic data of a virtual postoperative cornea, which has been treated by the predetermined refraction correction, is modeled for determining the aberration-neutral correction profile, wherein virtual wavefront aberration data is calculated for the virtual postoperative cornea the virtual topographic data and the beam passage model, wherein the predetermined refraction correction is adapted by a difference between the virtual wavefront aberration data of the virtual postoperative cornea and the wavefront aberration data of the preoperative cornea for providing the aberration-neutral correction profile.

6. The method according to claim 1, wherein a reference center is set for the topographic data, which is defined by a point of intersection of an axis of vision with a corneal surface, wherein the axis of vision extends from a central point of a pupil up to a fixation point external to eye.

7. The method according to claim 1, wherein predetermined corneal tomography data is additionally used for the calculation of the wavefront aberration data.

8. The method according to claim 1, wherein predetermined ocular aberration data is additionally used for the aberration-neutral correction profile.

9. A method for controlling a treatment apparatus, wherein the method comprises the following steps:
the method steps of a method according to claim 1, and
transferring the provided control data to a respective ophthalmological laser of the treatment apparatus.

10. A control device, which is configured to perform a respective method according to claim 1.

11. A treatment apparatus with at least one ophthalmological for the separation of a corneal volume with predefined interfaces of a human or animal eye by optical breakthrough, in particular by photodisruption and/or ablation, and at least one control device according to claim 10.

12. (canceled)

13. A computer-readable medium, on which a computer program according is stored, the computer program including commands, which cause a treatment apparatus to execute a method according to claim 1.
